(19) United States
(12) Patent Application Publication
(10) Pub. No.: US 2007/0255137 A1
(43) Pub. Date: Nov. 1, 2007

(54) Extended volume ultrasound data display and measurement

(75) Inventors: Lei Sui, Newcastle, WA (US); Arun Tirumalai, Sammamish, WA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/415,587

(22) Filed: May 1, 2006

(51) Int. Cl. A61B 8/00

(52) U.S. Cl. 600/443

(57) ABSTRACT

Three-dimensional ultrasound data acquisition is provided for extended field of view imaging or processing. The relative position of two or more three-dimensional volumes is determined using two-dimensional processes. For example, differences in position along two non-parallel planes are determined. By combining the vectors from the two differences, a relative position of the three-dimensional volumes is determined. Other features include calculating a value, such as a volume or distance, as a function of a relative position of two or more volumes, generating a two-dimensional extended field of view or multiplanar reconstruction as a function of a relative position without necessarily forming a three-dimensional extended field of view, and accounting for physiological phase for determining relative position or combining data representing different volumes.

### BACKGROUND

The present embodiments relate to three-dimensional imaging. In particular, three-dimensional ultrasound imaging of a large or elongated region of a patient is provided.

Commercially available ultrasound systems perform three-dimensional (3D) and four-dimensional (4D) volumetric imaging. Some 3D and 4D ultrasound systems use one-dimensional transducers to scan in a given plane. The transducer is translated or moved to various positions free-hand, resulting in a stack of planes with different relative spatial relationships representing a volume region of the patient. However, the relative position information and associated alignment of data may be inaccurate as compared to scans using multi-dimensional or wobbler transducers.

Using a volumetric imaging transducer, such as a multidimensional array or a wobbler transducer, ultrasound energy is transmitted and received along scan lines within a volume region or a region that is more than a two-dimensional plane within the patient. For some applications, the transducer geometry limits scanning to only a portion of the desired volume. For extended objects such as the liver or a fetus, the transducer scans only a section of the anatomical feature.

Extended field of view 3D and 4D imaging has been proposed. See U.S. Patent Application No. 2005/0033173, the disclosure of which is incorporated herein by reference. Two or more sets of data representing different volumes are combined together for imaging. The relative position of the volumes is determined from sensing a transducer position or data processing.

### BRIEF SUMMARY

By way of introduction, the preferred embodiments described below include methods and systems for three-dimensional ultrasound data acquisition for extended field of view three-dimensional processing or imaging. The relative position of two or more three-dimensional volumes is determined using two-dimensional processes. For example, differences in position along two non-parallel planes are determined. By combining the vectors from the two differences, a relative position of the three-dimensional volumes is determined. Other features include: calculating a value, such as a volume or distance, as a function of a relative position of two or more volumes, generating a two-dimensional extended field of view or multiplanar reconstruction as a function of a relative position without necessarily forming a three-dimensional extended field of view, and accounting for physiological phase for determining relative position or combining data representing different volumes. Any one or combination of two or more of these features may be used.

In a first aspect, a method is provided for three-dimensional ultrasound data acquisition. First and second sets of ultrasound data representing first and second three-dimensional volumes, respectively, of a patient are acquired with a volumetric imaging transducer. The first three-dimensional volume overlaps with but is different than the second three-dimensional volume. A relative position of the first and second three-dimensional volumes is determined. A value is calculated as a function of the relative position.

In a second aspect, a method is provided for three-dimensional ultrasound data acquisition. First and second sets of ultrasound data representing first and second three-dimensional volumes, respectively, of a patient are acquired with a volumetric imaging transducer. The first three-dimensional volume overlaps with but is different than the second three-dimensional volume. A first relative position of the first and second three-dimensional volumes is determined with at least two one and/or two-dimensional relative positions.

In a third aspect, a three-dimensional ultrasound data acquisition system is provided for extended field of view processing. A volumetric imaging transducer is operable to acquire first and second sets of ultrasound data representing first and second three-dimensional volumes, respectively, of a patient. The first three-dimensional volume overlaps with but is different than the second three-dimensional volume. A processor is operable to determine first and second relative positions of the first and second three-dimensional volumes along first and second two-dimensional planes, respectively.

In a fourth aspect, a method is provided for three-dimensional ultrasound data acquisition. First and second sets of ultrasound data representing first and second three-dimensional volumes, respectively, of a patient are acquired with a volumetric imaging transducer. The first three-dimensional volume overlaps with but is different than the second three-dimensional volume. A relative position of the first and second three-dimensional volumes relative to a physiological cycle is determined.

In a fifth aspect, a method is provided for three-dimensional ultrasound data acquisition. First and second sets of ultrasound data representing first and second three-dimensional volumes, respectively, of a patient are acquired with a volumetric imaging transducer. The first three-dimensional volume overlaps with but is different than the second three-dimensional volume. A relative position of the first and second three-dimensional volumes is determined. One or more two-dimensional extended field of view image from the ultrasound data of the first and second sets is generated as a function of the relative position.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components in the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound system for three-dimensional imaging;

FIG. 2 is a flow chart representing one embodiment of extended field of view three-dimensional imaging;

FIG. 3 is a graphical representation showing one embodiment of acquiring two volumes while translating a transducer;

FIG. 4 is a graphical representation of an extended field of view volume in one embodiment; and

FIG. 5 is a graphical representation of two-dimensional planes relative to three-dimensional volumes.

### DETAILED DESCRIPTION OF THE DRAWINGS AND PRESENTLY PREFERRED EMBODIMENTS

One or two-dimensional correlation, tracking or other position determining processes determine the relative position of three-dimensional volumes. Two-dimensional processes may be more computationally efficient than three-dimensional correlation or tracking. By performing one or two-dimensional correlation along different axes or two-dimensional planes, two or more degrees of freedom may be resolved. Two two-dimensional planes may be used to resolve six degrees of freedom—translation and rotation in three-dimensions.

The accuracy of one or two-dimensional position processing may result in an extended field of view for three-dimensions, providing accurate calculations. Determining relative positions for data associated with a same portion of a physiological cycle may provide more accuracy. The relative position is used to form a three-dimensional extended field of view and/or for forming two or more two-dimensional extended fields of view from two or more volumes. The three-dimensional extended field of view may allow for longer, more complex, more complete, and/or more thorough fly-through imaging of a volume.

FIG. 1 shows a block diagram of a medical diagnostic ultrasonic imaging system 10 for three- or four-dimensional processing. The three-dimensional processing includes determining relative positions, calculating values, or generating images. Three-dimensional imaging provides representations of a volume region as opposed to a planar region of a patient at a given time. Four-dimensional imaging provides a representation of a three-dimensional volume as a function of time, such as to show motion of features within the volume. The system 10 comprises any of now known or later developed ultrasound systems or workstations for three-dimensional processing or imaging.

The system 10 includes a transmit beamformer 12, a volumetric imaging transducer 14, a receive beamformer 16, an image processor 18, a 3D processor 20, a memory 22, and a display 24. Additional, different or fewer components may be provided. For example, ultrasound data is acquired from storage for processing in the 3D processor 20 without the transmit beamformer 12, the transducer 14, the receive beamformer 16 and/or the image processor 18. As another example, plane wave imaging may be used without beamformers 12, 14.

The transmit beamformer 12 includes memories, delays, amplifiers, waveform generators, oscillators, filters, modulators, analog devices, digital devices and combinations thereof for generating a plurality of waveforms in various channels. The waveforms are apodized and delayed relative to each other for electronic steering in either one or two dimensions, such as steering within a plane or steering within a volume or plurality of planes, respectively. Either full or sparse sampling may be provided, resulting in greater or fewer numbers of waveforms to generate for any given scan line. The transmit beamformer 12 applies the transmit waveforms to a volumetric imaging transducer 14.

The volumetric imaging transducer 14 is a multi-dimensional array, such as a two-dimensional array or other array of N by M elements where both N and M are greater than 1. By having a multi-dimensional array of elements, the volumetric imaging transducer 14 is operable to scan with scan lines electronically steerable in two dimensions, such as scanning a volume extending along any of three dimensions. Because of scanning along scan lines in two dimensions, multiple voxels are provided along any given azimuth, elevation and range dimension, resulting in a volumetric representation or scan.

In another embodiment, the volumetric imaging transducer 14 is a wobbler transducer. Any now known or later developed linear, one-dimensional array or single element is provided. The wobbler is mechanically steered in one or two dimensions and electrically steered in no or one dimension. In one embodiment, the scan lines are mechanically steered in one dimension, such as along an elevation dimension and electronically steered due to delays and apodization of waveforms in another dimension, such as the azimuth dimension. A wobbler array with electric steering in two dimensions may also be provided.

Other now known or later developed volumetric imaging transducers operable to acquire ultrasound data representing a volume with a greater extent than a planar slice of the patient may be used.

The volumetric imaging transducer 14 is operable to acquire a set of ultrasound data representing a three-dimensional volume of a patient. By directing scan lines at different positions within two dimensions, and receiving as a function of time representing the depth dimension, a three-dimensional volume may be scanned with the transducer 14 without movement of the transducer 14. Given the speed of sound through tissue, a volume is scanned even with movement of the transducer 14 by directing scan lines at different angles along the azimuth and elevation dimensions during translation. As a result, the volumetric imaging transducer 14 is used to acquire multiple sets of ultrasound data representing different three-dimensional volumes while stationary or while moving. The three-dimensional volumes overlap but represent different overall regions. In one embodiment, the overlap is just along the elevation dimension, but the transducer 14 may be moved along more than one axis and/or rotated, resulting in overlap along any of three dimensions.

Optionally, the transducer 14 includes a position sensor 26, such as a dedicated sensor for determining a position of the transducer 14 within a volume, area or adjacent to the patient. The sensor 26 is any now known or later developed magnetic, optical, gyroscope or other physical position measurement device. For example, electromagnetic coils positioned in the sensor are used to determine the position and orientation of the transducer 14 within a room. In alternative embodiments, the transducer 14 is free of the position sensor 26.

The receive beamformer 16 receives electrical signals generated by the transducer 14. The receive beamformer 16 has one or more delays, amplifiers, filters, demodulators, analog components, digital components and combinations thereof separated into a plurality of channels with a summer for summing the information from each of the channels. The summer or a subsequent filter outputs in-phase and quadrature or radio frequency data. Any now known or later developed receive beamformers may be used. The receive beamformer 16 outputs ultrasound data representing one or more scan lines to an image processor 18.

The image processor 18 is a digital signal processor, control processor, general processor, application specific integrated circuit, field programmable gate array, analog circuitry, digital circuitry or combination thereof. The image processor 18 detects intensity or B-mode information, estimates flow or Doppler information, or detects any other characteristic of the ultrasound data. The image processor may also implement temporal, spatial or frequency filtering. In one embodiment, the image processor 18 includes a scan converter, but a scan converter may be provided after the 3D processor 20 or as part of the 3D processor 20. One or more memories or buffers, such as a CINE memory, are optionally provided in the image processor 18. The image processor 18 outputs the detected ultrasound data to the 3D processor 20 in a polar coordinate, Cartesian coordinate or other format. Alternatively, the ultrasound data is output directly to the memory 22.

The 3D processor 20 is a general processor, digital signal processor, application specific integrated circuit, computer, field programmable gate array, video card, graphics processing unit, digital processor, analog processor, combinations thereof or other now known or later developed processor for processing and/or for generating a three-dimensional representation from data representing a volume region. In one embodiment, the 3D processor 20 is a processor used for or with other components of the system 10, such as a control processor for controlling the image processor 18. A separate or dedicated 3D processor 20 may be used.

The memory 22 is a RAM, buffer, portable, hard drive or other memory now known or later developed. In one embodiment, the memory 22 is part of another component of the system 10, such as CINE memory, a memory of the image processor 18 or a display plane memory, but a separate memory for three-dimensional processing may be provided.

The 3D processor 20 is operable to determine relative positions of three-dimensional volumes. The relative position is determined by reference to absolute positions, as an absolute position or differences between the positions of the volumes. In one embodiment, the 3D processor 20 receives position information from the sensor 26. In another embodiment, the 3D processor 20 determines relative position information from the ultrasound data. The data of one set is positionally related to the data of another set based on the relative positions of the transducer 14. The data representing one volume is spatially registered with the data representing another volume to form an extended volume. The 3D processor 20 may determine relative positions of three-dimensional volumes for 3D or 4D processes or imaging.

In one embodiment, three-dimensional correlation or tracking is performed. In another embodiment, the relative position is determined by one or two-dimensional processes, such as along two or more two-dimensional planes, respectively. A best or sufficient match of a two-dimensional region in one volume with a two-dimensional region of another volume provides translation and/or rotation between the two volumes with respect to the plane (e.g., two axes of translation and one axis of rotation). By determining translation and/or rotation along two non-parallel planes, different translation and/or rotation components are determined. The relative position includes any number of translation and/or rotation components.

A two-dimensional translation vector is determined for each plane. Alternatively, separate one-dimensional vectors are determined. Two such one-dimensional vectors define a plane.

The two non-parallel planes both extend, at least partly, into both volumes. Different planes may be used for determining relative position of different pairs or larger groupings of volumes. In one embodiment, a direction of motion of the transducer is determined, such as with three-dimensional correlation. In other embodiments, the direction is assumed. The two non-parallel planes each extend parallel to the direction of motion, but may be non-parallel with the direction of motion. The angular relationship of the two planes determines the geometric relationship of the different directional and rotations vectors. In other embodiments, only one plane extends into both volumes.

The two-dimensional planes may have any position relative to the transducer 14 and the volumes. In one embodiment, the two-dimensional planes are perpendicular to each other, such as one plane being a depth-azimuth plane and the other plane being an azimuth-elevation plane. As another example, the two planes are an azimuth-depth plane and an elevation-depth plane. The planes are at a center, edge or elsewhere relative to the volume. In one embodiment, more than one plane is used to determine a particular component of motion, such as using planes along the center of the volumes and parallel adjacent planes.

Ultrasound data used for determining position is all of or subsets of one or more of the volumes being combined. For example, data representing a likely overlapped area, such as associated with data adjacent to an edge of the volume in the direction of translation of a transducer 14 of one volume is compared with data likely overlapping of another volume. The data used may be further limited to data representing a portion or area of the two-dimensional planes. For example, data in areas of overlap representing the two non-parallel planes is used to determine position. In alternative embodiments, data from one of the sets of volume data is compared to data not used for three-dimensional imaging to determine the translation and associated positions of the transducer 14. Any combinations of data not used for three-dimensional imaging, data used for the three-dimensional imaging and combinations thereof may be used.

The data may be selected as a function of time. For example, sets of data representing the volumes are acquired over time. Due to physiological cycles, such as the heart or breathing cycle, the scanned volume may be different depending on when the volume was scanned. By gating or selecting ultrasound data associated with a substantially same time in the physiological cycle, the relative position and resulting extended volume may be more likely correct. A breath monitor, ECG, other device or analysis of ultrasound data may be used for identifying the temporal locations in the cycle.

The 3D processor 20 uses the relative position information to calculate a value associated with the different volumes. For example, a volume of a region which is not entirely represented in the component volumes, but is entirely represented in the extended volume is calculated based on the relative positions of the component volumes. Similarly, a distance, circumference or other value is calculated as a function of the relative position. Using the scan line density, pixel scale and/or known spatial distance of each voxel and the relative position of the volumes, accurate measurements may be made over the extended volume.

The 3D processor generates images from the ultrasound data representing the volumes. The images are generated as a function of the relative position. In one embodiment, a two-dimensional image is generated. The image corresponds to one of the planes used for determining the relative position or a different plane. The image is generated from data representing both volumes or the extended volume. For example, data from one volume is combined with data of another volume to form an extended field of view two-dimensional image without having combined data for a three-dimensional extended field of view.

For generating a two-dimensional image or three-dimensional representation, the ultrasound data representing one volume may be combined with ultrasound data representing a different volume, such as combining a first set with a second set. Alternatively, a subset of one, both or multiple of sets of ultrasound data representing different volumes are combined.

The combination is performed as a function of the relative positions of the volumes. The 3D processor 20 uses the combined data to generate a three-dimensional representation. The combined data is formatted in a polar coordinate, Cartesian or 3D grid. The data is interpolated or otherwise selected for rendering. Any of surface, projection, volume or other now known or later developed techniques for generating an image representing a three-dimensional volume may be used.

In one embodiment, multiplanar reconstruction images are generated. Two or more two-dimensional images (e.g., three images representing orthogonal planes) are generated with or without a three-dimensional representation. One or more of the two-dimensional images and/or the three-dimensional representation are generated as an extended field of view. The extended field of view is beyond the field of view available by a single scan volume.

The display 24 is a CRT, monitor, plasma screen, LCD, projector or other display device for generating an image representing the 3D volume. Using the 3D processor 20 and the display 24, the user may cause an image to be rotated or dissected for viewing the information within the three-dimensional volume from different angles or perspectives. In one embodiment, the 3D processor 20 and the display 24 are a separate workstation from the rest of the system 10, such as a workstation within the system 10 or remote from the system 10.

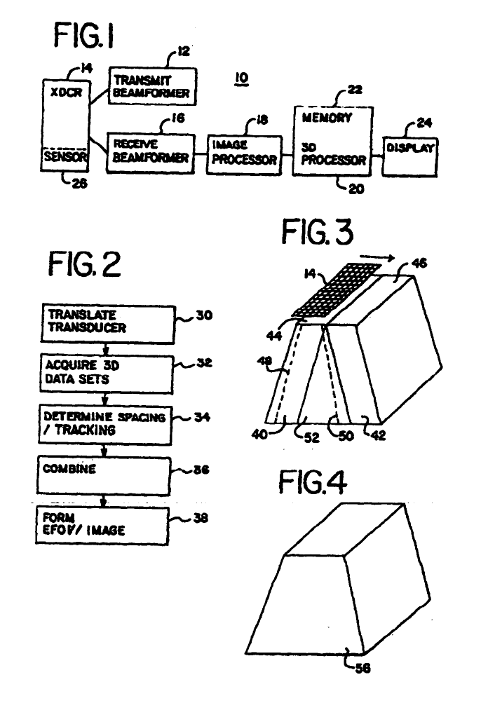

FIG. 2 shows a flow chart of a method for three-dimensional ultrasound acquisition and processing. The method of FIG. 2 is implemented using the system 10 of FIG. 1 or a different system. Additional, different or fewer acts may be provided. For example, the spacing or relative position is determined in act 34 without subsequent combination of act 36 and/or forming an extended field of view image of act 38. The relative position act 34 may be provided with additional acts for calculating a value.

In act 30, the transducer probe housing the transducer 14 is translated or moved between two different positions relative to the patient. In one embodiment, the transducer probe is slowly moved while different sets of data representing volumes are acquired. For example, the transducer is moved at about an inch per second so that ultrasound signals for 128 lines in 100 different slices are acquired for a given volume. Due to the speed of sound, the volume is acquired at a substantially same position of the transducer 14 even given the continuous movement of the transducer probe. Accordingly, multiple volumes are acquired at different transducer positions without ceasing movement of the transducer probe. Thirty or another number of volumes may be acquired each second, such as acquiring about 23 volumes a second for three seconds (total of about 70 volumes to be combined). More rapid or slower translation and associated scanning of a greater or lesser volume may be used. A sound or graphic may be provided to the user for indicating a desired translation speed. In alternative embodiments, the transducer probe is moved from one position to a second position and maintained at each of the positions for a time period, such as associated with acquiring ultrasound data for two different volumes through two different discrete acoustic windows.

The transducer 14 is moved free-hand. The user translates and/or rotates the transducer. Alternatively, a motor, mechanism, guide or robot moves the transducer 14.

The motion is along a particular axis, such as along the elevation or azimuth dimension. For example, the user translates the transducer 14 free-hand along an elevation dimension. The elevation dimension is defined by the transducer array. The transducer probe may be marked to indicate the elevation direction or array alignment. Alternatively, the transducer is moved in any direction.

In act 32, a plurality of ultrasound data sets representing a three-dimensional volume are acquired. For example, the data sets are acquired with the volumetric imaging transducer 14 while being translated over the patient. As shown in FIG. 3A, two volumes 40 and 42 are acquired associated with translating the transducer 14 from or through a position 44 to or past the position 46. As a result, the ultrasound data representing the volume 40 overlaps with the ultrasound data representing the volume 42. While the transducer positions 44 and 46 do not overlap, some overlap may be provided or the positions may be further separated.

For 4D imaging or processing, multiple sets of three-dimensional volume sets are acquired. The three-dimensional acts may be applied for four-dimensional processing.

Acoustic energy is steered from the transducer 14 at two or more different angles relative to the transducer 14 to scan each volume 40, 42. As shown by the scan lines 48 and 50, two of the different angles used are along a dimension substantially parallel to the direction- of translation. Any number of scan lines and associated angles may be used. As a result of the different angles along the direction of translation as well as along another dimension, data representing a volume is acquired. Alternatively, linear or orthogonal scan lines are used.

As discussed above, the ultrasound data representing the first volume 40 is acquired with the transducer 14 held at a stationary position 44 or as the transducer 14 is translated without stopping through the position 44. Likewise, the ultrasound data representing the second volume 42 is acquired with the transducer held in the position 46 or as the transducer 14 is translated through the position 46. Where the transducer 14 is held substantially stationary, substantially is provided to account for movement due to breathing, heart motion or unintentional movement of the sonographer or patient. Where the volumes 40, 42 are acquired while translating the transducer 14 without stopping at each position, the sets of data are acquired sufficiently rapidly in comparison to the rate of translation of the transducer to allow acquisition of the volume. Where the translation of the transducer 14 causes a perceived compression of the data, interpolation, morphing or other techniques may be used to account for motion of the transducer 14 in acquisition of data throughout the volume.

As shown in FIG. 3, a portion of ultrasound data representing each of the volumes 40 and 42 corresponds to an overlapping region 52. Data from each of the volumes 40 and 42 represent the overlapping region 52. The data may or may not occur at the identical spatial location within the overlapping region 52.

While only two volumes 40 and 42 are shown, additional volumes with more or less overlap may be provided, including an initial volume and an ending volume with no overlap. The overlap shown in FIG. 3 is associated with transducer positions 44 and 46 along one dimension, such as the elevation dimension. Rotation and translation along other or additional dimensions relative to the transducer 14 array may be provided.

The acquired ultrasound data is left in a same polar coordinate or Cartesian coordinate format. Alternatively, the data representing the volumes is reformatted onto a 3D grid that is common for all volumes. The ultrasound data representing the various three-dimensional volumes of the patient is stored. In one embodiment, each of the sets of data representing a different volume is stored separately. In alternative embodiments, the ultrasound data is combined and then stored after combination.

In act 34, a relative position or spacing of the first position 44 to the second position 46 is determined. The positioning is determined within the three dimensional space accounting for translation and rotation. Alternatively, positions along a single dimension without rotation or positions corresponding to any number of translational and/or rotational degrees of freedom are determined.

In one embodiment, the position of the volumetric imaging transducer 14 is tracked using ultrasound data. The relative spacing between the two positions is determined from the ultrasound data. The ultrasound data used for the tracking is the data from one, both, or different data than the sets of data representing the three-dimensional volumes. Filtering, correlation, the sum of absolute differences, decorrelation or other techniques are used for identifying and registering speckle or features from one data set in a different data set. For example, speckle or features are isolated and used to determine a pattern from one set of data that is most similar to a pattern of another set of data. The amount of translation and rotation of the best pattern match provides a vector representing translation and identifies a rotation. In one embodiment, a pattern based on a subset of the ultrasound data of one volume is used for matching with another set. Alternatively, multiple subsets of data representing spatially different volumes or planes along different dimensions are used for the pattern matching. Alternatively, all of the data of one data set is pattern matched with all of the data of another data set. As yet another alternative, sub-sampling of the entire data set or portions of a data set are used to match against a sub-sampling or full sampling of another data set.

Any of various now known or later developed two-or three-dimensional techniques for determining positions from the data may be used, such as disclosed in U.S. Pat. Nos. 5,876,342, 5,575,286, 5,582,173, 5,782,766, 5,910,114, 5,655,535, 5,899,861, 6,059,727, 6,014,473, 6,171,248, 6,360,027, 6,364,835, 6,554,770, 6,641,536 and 6,872,181, the disclosures of which are incorporated herein by reference. Any of the two-dimensional correlation, decorrelation, or motion tracking techniques discussed in the patents above or now known or later developed may be used or expanded for correlation and tracking of speckle or features in a three-dimensional volume or using a three-dimensional data set for the correlations or other calculations. For speckle tracking, decorrelation or correlation is determined. For feature tracking, a sum of absolute differences is determined. In one embodiment, both speckle and feature information are tracked and the combined translation and rotation information, such as an average, is used. Since additional speckle and structural information is provided in a three-dimensional volume as opposed to a planar image, the registration of one volume relative to another volume may be more accurate and accomplish more degrees of freedom rather than relying on an elevation speckle decorrelation in a two-dimensional image.

The determined translation and rotation or registration information provides the relative positions between various transducer positions 44 and 46 for acquiring ultrasound data representing the different volumes. The position information also provides relationship information for various voxels within the overlapping region 52.

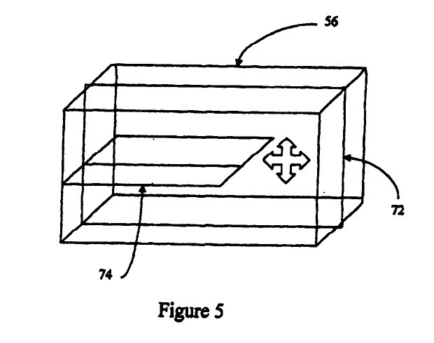

In one embodiment of act 34, the relative position is determined as a function of tracking along two non-parallel two-dimensional planes or three lines or axes. FIG. 5 shows the extended volume 56. For ease of reference, the extended volume 56 is not shown as separate overlapping volumes, such as represented in FIG. 3. The extended volume 56 of FIG. 5 corresponds to separate, overlapping volumes. The planes correspond to acquired image planes or other planes. For other planes, the ultrasound data may be interpolated, extrapolated, synthesized or combined to provide ultrasound date representing the planes.

Two planes 72, 74 are defined. The planes 72, 74 are predetermined relative to the transducer, an expected direction of motion, a determined direction of motion, arbitrary or other relationship. One or both planes 72, 74 extend into, at least in part, two or more volumes. In FIG. 5, the two planes 72, 74 are orthogonal or perpendicular, but other non-parallel relationships may be used. A line formed by the intersection of the two planes extends substantially parallel with an intended direction of motion of the transducer. Alternatively, the intersection line is arbitrary in position or extends along a depth direction, such as both planes 72, 74 and the line being along a center depth axis in one of the volumes. The planes are parallel with dimensions of the transducer, such as the plane 72 being in a depth-elevation plane (elevation direction) and the plane 74 being in an azimuth-elevation plane (lateral direction). Alternatively, one or both planes have one or both dimensions which are non-parallel with one or more of the transducer dimensions. Any positioning of the non-parallel planes may be used.

More than two planes may be used. For example, two planes are centered through at least one volume. Additional planes in parallel with or non-parallel to the two other planes are also defined and used for position determination. For example, groups of two or three parallel planes are used. The groups may have any spacing, such as being near a center, near an edge or distributed in any pattern in between.

A displacement vector or relative position is determined for each of the planes. For example, two or more two-dimensional relative positions are determined, one for each plane. Any now known or later developed two-dimensional tracking or position determination may be used. For example, correlation (e.g., sum of absolute differences or cross-correlation) between a region along a plane selected from one volume with a search region along the plane in another volume is performed. The data for the region is compared in different relative positions to data of the search region to identify a highest or sufficient correlation. The relative position of the region with the best fit in the search region provides a two-dimensional vector with or without rotational matching providing a rotational component.

In one embodiment, the region is divided into a plurality of sub-regions. Each sub-region is correlated with the search region. A global relative position is calculated, such as from an average, from the sub-region vectors. Such processes are described in U.S. Pat. Nos. 5,899,861, 5,575,286 or other ones of the patents cited above.

The displacement vector or relative position along each plane is determined. The vectors are combined to determine a three-dimensional relative position.

In one embodiment, one or more of the volumes is subject to physiological cycle variation as a function of time. The ultrasound data is obtained for a specific portion or portions of the physiological cycle. The relative positions are determined using ultrasound data associated with a same portion of the physiological cycle. The speckle and/or features used for matching or correlation are more likely similar where the position is determined relative to a physiological cycle.

In an alternative embodiment of act 34, the relationship between the different positions 44 and 46 of the volumetric imaging transducer 14 is provided by a sensor 26 on the transducer 14. The sensor 26 mounted on the transducer 14 provides an absolute position within a room or volume or provides a difference in position from a previous position, such as providing an amount of motion and direction as a function of time. In either case, the difference in translation and/or rotation between two different transducer positions 44, 46 and the associated spatial relationship of the ultrasound data representing the volumes 40 and 42 is determined.

In optional act 36, different sets of ultrasound data representing the different volumes are combined. Each set of ultrasound data is aligned relative to other sets of data as a function of the determined spacing or relative position of act 34 for combination. The two volumes 40 and 42 shown in FIG. 3 are aligned as shown and combined to form the volume 56 shown in FIG. 4.

In the overlapping region 52, the ultrasound data from the first set is compounded with the ultrasound data from the second set, such as averaging or weighted averaging. Any of various combination techniques may be used, such as selecting a maximum or minimum value, or adaptively compounding as a function of amount of correlation, type of data, signal-to-noise ratio of data or other parameters determined from the ultrasound data or the sensor 26. In one embodiment, a finite impulse response filtering with an equal weighted averaging of one or more values associated with a particular location on a 3D grid from any or all sets of data overlapping at that location is performed. For example, the nearest four pixel values to a 3D grid point for each set of data are weighted as a function of the distance of the data value from the grid point with equal or spatially related weighting being applied between the sets of data. The resulting compounded values are normalized. Any of various now known or later developed interpolation and compounding techniques may be used. In alternative embodiments, interpolation to the 3D grid and combination of ultrasound data from different data sets is performed separately.

Regions where only one set of data represents the region are included in the combination without averaging or other alteration. Alternatively, these regions are either removed or the ultrasound data is increased or decreased to account for processing of the overlapped regions to avoid stripes or differences in gain. In one embodiment avoiding compounding in the combination, ultrasound data from only non-overlapping regions are added to the ultrasound data set of another volume, such as growing the combined volume without compounding data points from different sets presenting a same or substantially same spatial location.

In one embodiment, the ultrasound data representing a feature or a volume in general is morphed or altered as a function of pressure distortion prior to combination. In alternative embodiments, the morphing occurs after combination. For example, the ultrasound data is interpolated to account for pressure, such as caused by the transducer compressing or warping an organ while being placed on the skin or caused by heart cycle pressure placed on the organ.

In optional act 38, a three-dimensional representation image responsive to the combined ultrasound data is formed or generated. For example, a maximum intensity projection, minimum intensity projection, weighted projection, or alpha blending is volume rendered for one or a plurality of different look directions relative to the volume 56. Alternatively, a surface rendering with or without associated shading is generated as an image. Any of various now known or later developed three-dimensional imaging techniques given ultrasound data representing the volume may be used.

The displayed three-dimensional representation provides an extended field of view. Rather than providing a three-dimensional image based on each of the volumes 40 and 42 separately, a three-dimensional image representing the combined volume 56 is provided. This extended field of view in three-dimensions is larger than a region or view acquired with the transducer 14 held stationary. In one embodiment, the ultrasound data for the entire combined region 56 is used to generate the three-dimensional representation. Alternatively, ultrasound data of selected portions of the combined region 56 is used, such as only using a first portion of either the first volume 40 or second volume 42. For the extended field of view, at least a portion of one of the data sets is included for generating a three-dimensional representation with data from the other data set.

In another embodiment of act 38, a two-dimensional extended field of view image is generated from the ultrasound data from the different volumes or a combined, extended field of view volume. The two-dimensional extended field of view image is generated as a function of the relative position. The plane of the image is one of the planes used to determine relative position or a different plane. The plane of the image corresponds to one or more scan planes or ultrasound data is interpolated, extrapolated or synthesized to the desired plane. Using the relative position, data from different sets or volumes may contribute to the two-dimensional field of view. The data is compounded or selected for each pixel location. For example, data from different volumes is combined as discussed above for act 36, only just along the image plane. As another example, data from a combined volume is selected.

One or more two-dimensional extended fields of view may be generated. For example, a multiplanar reconstruction is performed for the extended volume. Two or more two-dimensional images representing different cross-sections or slices through the extended volume are generated and displayed substantially simultaneously. The two-dimensional images may be displayed with one or more three-dimensional representations of the extended volume or one or more of the component volumes.

In another embodiment, a value is calculated as a function of the relative position. Various spatial calculations are a function of data outside of one of the component volumes, such as making use of the extended volume. For example, a volume of a region entirely within the extended volume but not entirely within any of the component volumes is calculated. As another example, a distance from a first point not within one three-dimensional volume to a second point not within another three-dimensional volume is calculated. Other calculations include boundary detection calculations or circumference.

The spatial calculation is a function of the voxel size or region represented by each ultrasound value. For example, the scan line density, size of the array or other information is used to determine the pixel or voxel scale. The relative position spatially aligns data from one volume to data from another volume, allowing spatial calculations. The spatial calculations are performed with ultrasound data from separate data sets, such as from uncombined volume sets, or from ultrasound data combined to represent an extended volume.

While described above for two volumes generally, three or more volumes may be combined as discussed herein. Multiple volumes are spliced together to visualize larger organs as one composite volume and may provide different levels of compounding. The composite volume may be reacquired multiple times to provide an extended field of view 4D imaging (i.e. 3D imaging with the composite volume as a function of time). The 3D applications described herein may be used for 4D imaging or processing.

A composite volume three-dimensional representation may be displayed while acquiring multiple three-dimensional representations. Other displays representing either a component volume or the combined volume, such as an arbitrary slice through the volumes, may be generated before a final display. Other two-dimensional images may be displayed while acquiring the component volume sets of data or while displaying the compounded or composite three-dimensional representation. The extended field of view three-dimensional representation is used for 3D surgical planning and/or fly through analysis. Four-dimensional functional or panoramic images information may be detected and displayed, such as imaging with strain information or contrast agent perfusion, inflow or outflow information within or as the compound volume three-dimensional representation. B-mode, Doppler velocity, Doppler power, or other types of information are used independently or together for the display of the three-dimensional representation. For example, a power mode Doppler display is generated without B-mode information from Doppler data acquired for multiple volumes. As another example, strain, strain rate or other parametric imaging formats are used for extended field of view three-dimensional processing or imaging.

Other imaging modalities may be used to generate a large field of view volume image or a data set representing the large, 3D field of view. Other imaging modalities may include computed tomography, x-ray, magnetic resonance, or positron emission. The extended field of view generated with ultrasound data may be responsive to the data of the other imaging modality. In real-time or offline, the data representing a volume or images from other modalities is used to calibrate the geometry of the ultrasound extended field of view. For example, the relative position for the ultrasound volumes is refined or a function of data from another modality. As another example, the combination of data from overlapping volumes is a function of the data from another modality. In additional or alternative embodiments, data from different modalities representing a same, similar or overlapping field of view are combined. Calibration or fusing data from different modalities may assist in surgical guidance or planning or diagnosis.

While the invention has been described above by reference to various embodiments, it should be understood that many changes and modifications can be made without departing from the scope of the invention. For example, for real time tracking with minimal processing, the user is instructed to translate along one dimension and the motion is tracked just along one dimension, such as the elevation dimension. It is therefore intended that the foregoing detailed description be regarded as illustrative rather than limiting, and that it be understood that it is the following claims, including all equivalents, that are intended to define the spirit and scope of this invention.

I (we) claim:

1. A method for three-dimensional ultrasound data acquisition, the method comprising:
acquiring first and second sets of ultrasound data representing first and second three-dimensional volumes, respectively, of a patient with a volumetric imaging transducer, the first three-dimensional volume overlapping with but different than the second three-dimensional volume;
determining a relative position of the first and second three-dimensional volumes; and
calculating a value as a function of the relative position.

2. The method of claim 1 further comprising:
combining ultrasound data from the first set with ultrasound data from the second set as a function of the relative position; and
generating a three-dimensional representation image responsive to the combined ultrasound data, wherein the three-dimensional representation image represents both of the first and second three-dimensional volumes including at least a first portion of the first three-dimensional volume outside the second three-dimensional volume and at least a second portion of the second three-dimensional volume outside the first three-dimensional volume.

3. The method of claim 1. wherein acquiring comprises moving, free-hand, the volumetric imaging transducer between a first position associated with the first three-dimensional volume and a second position associated with the second three-dimensional volume.

4. The method of claim 1 wherein calculating the value comprises calculating a volume value for a region not entirely within the first or the second three-dimensional volumes.

5. The method of claim 1 wherein calculating the value comprises calculating as a function of a distance from a first point not within the second three-dimensional volume to a second point not within the first three-dimensional volume.

6. The method of claim 1 wherein determining the relative position comprises determining as a function of ultrasound data part of or separate from the first and second sets.

7. The method of claim 6 wherein determining comprises determining the relative position of the first and second three-dimensional volumes as a function of tracking along two non-parallel two-dimensional planes.

8. The method of claim 1 wherein determining the relative position comprises determining relative to a physiological cycle.

9. The method of claim 1 further comprising generating a two-dimensional extended field of view image from the ultrasound data of the first and second sets as a function of the relative position.

10. A method for three-dimensional ultrasound data acquisition, the method comprising:
acquiring first and second sets of ultrasound data representing first and second three-dimensional volumes, respectively, of a patient with a volumetric imaging transducer, the first three-dimensional volume overlapping with but different than the second three-dimensional volume;
determining a first relative position of the first and second three-dimensional volumes with at least two two-dimensional relative positions.

11. The method of claim 10 wherein determining comprises:
determining a second relative position of the first and second three-dimensional volumes along a first two-dimensional plane;
determining a third relative position of the first and second three-dimensional volumes along a second two-dimensional plane, the second two-dimensional plane non-parallel with the first two-dimensional plane; and
determining the first relative position of the first and second three-dimensional volumes as a function of the second and third relative positions.

12. The method of claim 11 wherein determining the second and third relative positions comprises determining with the first two-dimensional plane perpendicular to the second two-dimensional plane, at least one of the first and second two-dimensional planes extending into both the first and second volumes.

13. The method of claim 12 wherein the first two-dimensional plane is along an elevation direction and the second two-dimensional plane is along a lateral direction relative to the volumetric imaging transducer.

14. The method of claim 11 wherein the first and second two-dimensional planes substantially pass through a center depth axis in the first volume.

15. The method of claim 10. wherein determining the first relative position comprises determining as a function of ultrasound data part of or separate from the first and second sets.

16. The method of claim 10 further comprising:
calculating a value as a function of the first relative position.

17. The method of claim 10 further comprising:
combining ultrasound data from the first set with ultrasound data from the second set as a function of the first relative position; and
generating a three-dimensional representation image responsive to the combined ultrasound data, wherein the three-dimensional representation image represents both of the first and second three-dimensional volumes including at least a first portion of the first three-dimensional volume outside the second three-dimensional volume and at least a second portion of the second three-dimensional volume outside the first three-dimensional volume.

18. The method of claim 10 wherein determining the first relative position comprises determining relative to a physiological cycle.

19. The method of claim 10 further comprising generating a two-dimensional extended field of view image from the ultrasound data of the first and second sets as a function of the first relative position.

20. A three-dimensional ultrasound data acquisition system for extended field of view processing, the system comprising:
a volumetric imaging transducer operable to acquire first and second sets of ultrasound data representing first and second three-dimensional volumes, respectively, of a patient, the first three-dimensional volume overlapping with but different than the second three-dimensional volume; and
a processor operable to determine first and second relative positions of the first and second three-dimensional volumes along first and second two-dimensional planes, respectively.

21. The system of claim 20 wherein the volumetric imaging transducer comprises a multi-dimensional array operable to scan with scan lines steerable in two dimensions or a wobbler transducer operable to scan with scan lines steerable in two dimensions.

22. A method for three-dimensional ultrasound data acquisition, the method comprising:
acquiring first and second sets of ultrasound data representing first and second three-dimensional volumes, respectively, of a patient with a volumetric imaging transducer, the first three-dimensional volume overlapping with but different than the second three-dimensional volume; and
determining a relative position of the first and second three-dimensional volumes relative to a physiological cycle.

23. A method for three-dimensional ultrasound data acquisition, the method comprising:
acquiring first and second sets of ultrasound data representing first and second three-dimensional volumes, respectively, of a patient with a volumetric imaging transducer, the first three-dimensional volume overlapping with but different than the second three-dimensional volume;
determining a relative position of the first and second three-dimensional volumes; and
generating two or more two-dimensional extended field of view image from the ultrasound data of the first and second sets as a function of the relative position.

24. The method of claim 23 wherein generating comprises generating a multiplanar reconstruction view.
